(19) United States
(12) Patent Application Publication
(10) Pub. No.: US 2026/0278833 A1
(43) Pub. Date: Sep. 17, 2026

(54) IMAGE PROCESSING APPARATUS, IMAGE PROCESSING METHOD, AND IMAGE PROCESSING PROGRAM

(71) Applicant: OLYMPUS MEDICAL SYSTEMS CORP., Tokyo (JP)

(72) Inventors: Masanori MITSUI, Tokyo (JP); Yasuko IKEGAMI, Tokyo (JP); Munenori FUKUNISHI, Tokyo (JP); Kosuke MOTAI, Saitama (JP)

(73) Assignee: OLYMPUS MEDICAL SYSTEMS CORP., Tokyo (JP)

(21) Appl. No.: 19/371,415

(22) Filed: Oct. 28, 2025

Related U.S. Application Data
(63) Continuation of application No. PCT/JP2023/024564, filed on Jul. 3, 2023.

(51) Int. Cl. G06T 7/73; A61B 34/20; G06T 7/13; G16H 30/40

(52) U.S. Cl. CPC G06T 7/74; A61B 34/20; G06T 7/13; G16H 30/40; A61B 2034/2065; G06T 2207/10068; G06T 2207/30096; G06T 2207/30244

(57) ABSTRACT

An image processing apparatus includes a processor that generates a three-dimensional image of a subject from a first image acquired by an endoscope before an acquisition of a second image, associates a position of boundary information of a lesion included in the second image with the second image, and associates the position of the boundary information associated with the second image with the three-dimensional image. The first and second images are two-dimensional images of the subject. The processor: calculates a relative position between a treatment tool in a two-dimensional third image acquired by the endoscope after the acquisition of the second image and a camera of the endoscope; calculates a position of the camera in the three-dimensional image; calculates a position of the treatment tool in the three-dimensional image; determines a positional relationship between the treatment tool and the boundary information; and outputs support information based on a determination result.

### CROSS-REFERENCE TO RELATED APPLICATIONS

This is a continuation of International Application PCT/JP2023/024564 which is hereby incorporated by reference herein in its entirety.

### TECHNICAL FIELD

The present invention relates to an image processing apparatus, an image processing method, and an image processing program.

### BACKGROUND ART

Conventionally, there are known systems for supporting endoscopic procedures (for example, see PTLs 1 and 2) . In PTL 1, an image showing the distal end of a treatment tool and the distance from the distal end of the treatment tool to a cut site are overlaid on a three-dimensional image of an organ, and a composite three-dimensional image is displayed. In PTL 2, the shape of a luminal organ, such as the large intestine, is calculated from a three-dimensional image, the shape of an endoscope is calculated, and the observation position of the endoscope in the organ is estimated based on the correspondence between the shape of the endoscope and the shape of the organ.

### CITATION LIST

### Patent Literature

{PTL1} Japanese Unexamined Patent Application, Publication No. 2013-202313
{PTL2} Japanese Unexamined Patent Application, Publication No. 2014-230612

### SUMMARY OF INVENTION

An aspect of the present invention is an image processing apparatus that processes an image acquired by an endoscope, the apparatus including a processor configured to: generate a three-dimensional image of a subject from a first image; associate a position of boundary information of a lesion included in a second image with the second image; and associate the position of the boundary information associated with the second image with the three-dimensional image. The first image and the second image are two-dimensional images of the subject acquired by the endoscope, and the first image is acquired before an acquisition of the second image. The processor is further configured to: calculate a relative position between a treatment tool in a third image and a camera of the endoscope based on the third image, the third image being a two-dimensional image acquired by the endoscope after the acquisition of the second image; calculate a position of the camera in the three-dimensional image based on the third image; calculate a position of the treatment tool in the three-dimensional image based on the relative position and the position of the camera in the three-dimensional image; determine a positional relationship between the treatment tool and the boundary information based on the position of the boundary information associated with the three-dimensional image and the position of the treatment tool in the three-dimensional image; and output support information based on a determination result.

Another aspect of the present invention is an image processing apparatus that processes an image acquired by an endoscope, the apparatus including a processor configured to: generate a three-dimensional image of a subject from a first image; associate a position of boundary information of a lesion included in a second image with the second image; and associate the position of the boundary information associated with the second image with the three-dimensional image. The first image and the second image are two-dimensional images of the subject acquired by the endoscope, and the first image is acquired before an acquisition of the second image. The processor is further configured to: associate the position of the boundary information with the second image by detecting the boundary information in the second image, the second image having a wide field of view including an entirety of the lesion; associate the position of the boundary information in the second image with a third image via the three-dimensional image, the third image being a two-dimensional image acquired by the endoscope after the acquisition of the second image and being a two-dimensional image having a narrower field of view than the second image; calculate the position of the boundary information outside the field of view of the third image; and output the third image together with navigation information relating to the boundary information outside the field of view.

Another aspect of the present invention is an image processing apparatus that processes an image acquired by an endoscope, the apparatus including a processor configured to: generate a three-dimensional image of a subject from a first image; associate a position of boundary information of a lesion included in a second image with the second image; and associate the position of the boundary information associated with the second image with the three-dimensional image. The first image and the second image are two-dimensional images of the subject acquired by the endoscope, and the first image is acquired before an acquisition of the second image. The boundary information is a line to be treated by a treatment tool. The processor is further configured to: determine whether or not the treatment tool in a third image is performing a treatment, the third image being a two-dimensional image acquired by the endoscope after the acquisition of the second image; calculate, when it is determined that the treatment tool is performing a treatment, a position of the treatment tool in the three-dimensional image at that time based on the third image; determine progress of the treatment based on a position of the line associated with the three-dimensional image and the position of the treatment tool in the three-dimensional image; and output the third image together with progress information indicating the progress of the treatment.

Another aspect of the present invention is an image processing method for processing an image acquired by an endoscope. The image processing method includes: generating a three-dimensional image of a subject from a first image; associating a position of boundary information of a lesion included in a second image with the second image; and associating the position of the boundary information associated with the second image with the three-dimensional image. The first image and the second image are two-dimensional images of the subject acquired by the endoscope, and the first image is acquired before an acquisition of the second image. The method further includes: calculating a relative position between a treatment tool in a third image and a camera of the endoscope based on the third image, the third image being a two-dimensional image acquired by the endoscope after the acquisition of the second image; calculating a position of the camera in the three-dimensional image based on the third image; calculating a position of the treatment tool in the three-dimensional image based on the relative position and the position of the camera in the three-dimensional image; determining a positional relationship between the treatment tool and the boundary information based on the position of the boundary information associated with the three-dimensional image and the position of the treatment tool in the three-dimensional image; and outputting support information based on a determination result.

Another aspect of the present invention is a non-transitory computer-readable recording medium having an image processing program stored therein, the program causing a processor to execute an image processing method for processing an image acquired by an endoscope, the image processing method including: generating a three-dimensional image of a subject from a first image; associating a position of boundary information of a lesion included in a second image with the second image; and associating the position of the boundary information associated with the second image with the three-dimensional image. The first image and the second image are two-dimensional images of the subject acquired by the endoscope, and the first image is acquired before an acquisition of the second image. The method further includes: calculating a relative position between a treatment tool in a third image and a camera of the endoscope based on the third image, the third image being a two-dimensional image acquired by the endoscope after the acquisition of the second image; calculating a position of the camera in the three-dimensional image based on the third image; calculating a position of the treatment tool in the three-dimensional image based on the relative position and the position of the camera in the three-dimensional image; determining a positional relationship between the treatment tool and the boundary information based on the position of the boundary information associated with the three-dimensional image and the position of the treatment tool in the three-dimensional image; and outputting support information based on a determination result.

### BRIEF DESCRIPTION OF DRAWINGS

FIG. 1 illustrates the overall configuration of an endoscope system according to a first embodiment.

FIG. 2A is a flowchart of an image processing method according to the first embodiment.

FIG. 2B is a flowchart for explaining an example of step S6 in the image processing method in FIG. 2A.

FIG. 2C is a flowchart for explaining another example of step S6 in the image processing method in FIG. 2A.

FIG. 3 illustrates steps in a gastric ESD.

FIG. 4A illustrates a sequence of first images.

FIG. 4B illustrates a three-dimensional image generated from the sequence of first images.

FIG. 4C illustrates a three-dimensional image associated with boundary information.

FIG. 5A illustrates boundary information associated with a second image.

FIG. 5B illustrates a third image including a treatment tool.

FIG. 5C illustrates a third image for explaining an example of support information.

FIG. 6A illustrates an example of a third image with a near field.

FIG. 6B illustrates an example of navigation information and a composite image.

FIG. 7 is a flowchart of an image processing method according to a second embodiment.

FIG. 8A is a flowchart for explaining a first example of steps S15 and S16 in the image processing method in FIG. 7.

FIG. 8B illustrates the image processing in FIG. 8A.

FIG. 9A is a flowchart for explaining a second example of steps S15 and S16 in the image processing method in FIG. 7.

FIG. 9B illustrates the image processing in FIG. 9A.

FIG. 10A is a flowchart for explaining a third example of steps S15 and S16 in the image processing method in FIG. 7.

FIG. 10B illustrates the image processing in FIG. 10A.

FIG. 11 is a flowchart of an image processing method according to a third embodiment.

FIG. 12A illustrates an example of progress information.

FIG. 12B illustrates another example of progress information.

FIG. 12C illustrates another example of progress information.

FIG. 12D illustrates another example of progress information.

### DESCRIPTION OF EMBODIMENTS

### First Embodiment

An image processing apparatus, an image processing method, and an image processing program according to a first embodiment of the present invention will be described with reference to the drawings.

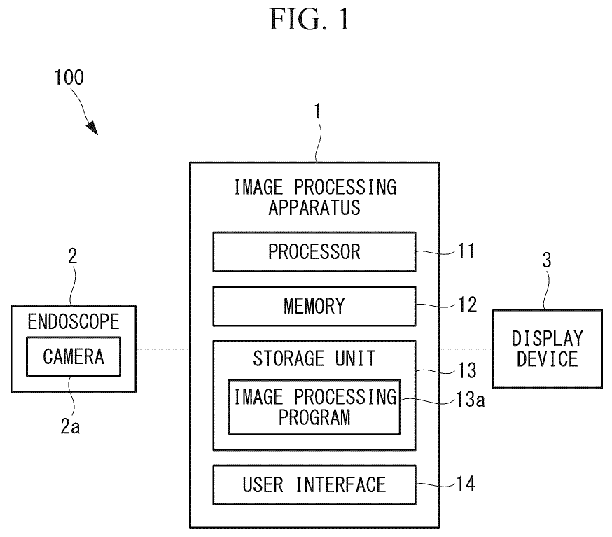

As illustrated in FIG. 1, an image processing apparatus 1 according to this embodiment is applied to an endoscope system 100.

The endoscope system 100 includes an endoscope 2, the image processing apparatus 1 that processes an image acquired by the endoscope 2, and a display device 3.

The endoscope 2 is a typical monocular endoscope and has a channel (not illustrated) through which a treatment tool 4 is inserted. The endoscope 2 has a two-dimensional camera 2a, including an imaging device and an objective lens, at the distal end thereof. A two-dimensional image of a subject photographed by the camera 2a is input from the endoscope 2 to the display device 3 through the image processing apparatus 1, and is displayed on the display device 3.

The image processing apparatus 1 generates support information for supporting an appropriate operation of the treatment tool 4 by a surgeon from two-dimensional images acquired by the endoscope 2 before and during an endoscopic procedure, and outputs the support information.

Specifically, the image processing apparatus 1 includes a processor 11, such as a central processing unit, a memory 12, such as a random access memory (RAM), a storage unit 13, and a user interface 14.

The storage unit 13 is a non-transitory computer-readable recording medium, such as a hard disk drive or a read-only memory (ROM). The storage unit 13 stores an image processing program 13a for causing the processor 11 to execute an image processing method described below. The storage unit 13 also stores data, such as optical design data of the endoscope 2, necessary for image processing.

The user interface 14 includes an input device, such as a mouse, a keyboard, and a touch panel. A user, such as a surgeon, can input information to the image processing apparatus 1 through the user interface 14 by operating the input device.

Next, an image processing method executed by the processor 11 will be described.

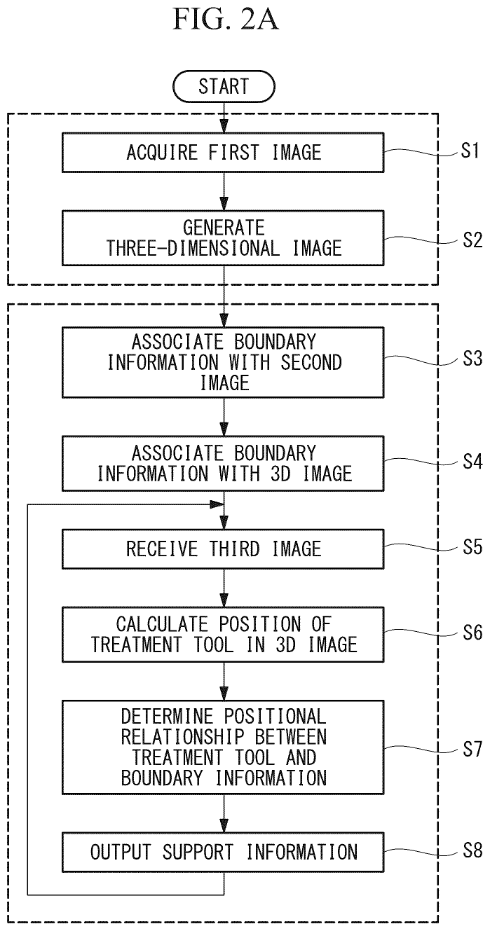

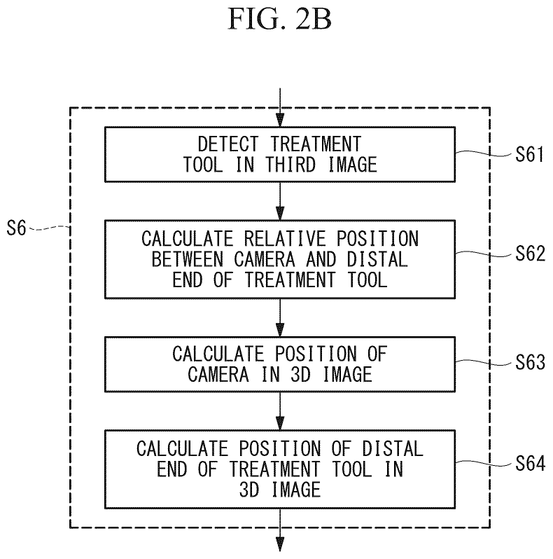

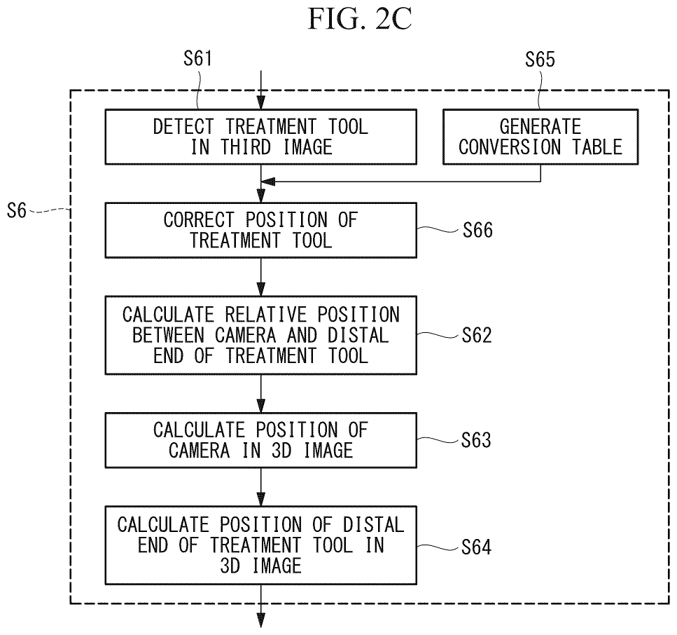

As illustrated in FIG. 2A, the image processing method according to this embodiment includes step S1 of acquiring first images A of a subject S, step S2 of generating a three-dimensional (3D) image D of the subject S from the first images A, step S3 of associating the position of boundary information E of a lesion T with a second image B of the subject S, step S4 of associating the position of the boundary information E with the 3D image D, step S5 of receiving a third image C of the subject S, step S6 of calculating the position of the treatment tool 4 in the 3D image D based on the third image C, step S7 of determining the positional relationship between the treatment tool 4 and the boundary information E, and step S8 of outputting support information based on the determination result.

Steps S1 and S2 are performed before an endoscopic procedure, and steps S3 to S8 are performed during the endoscopic procedure.

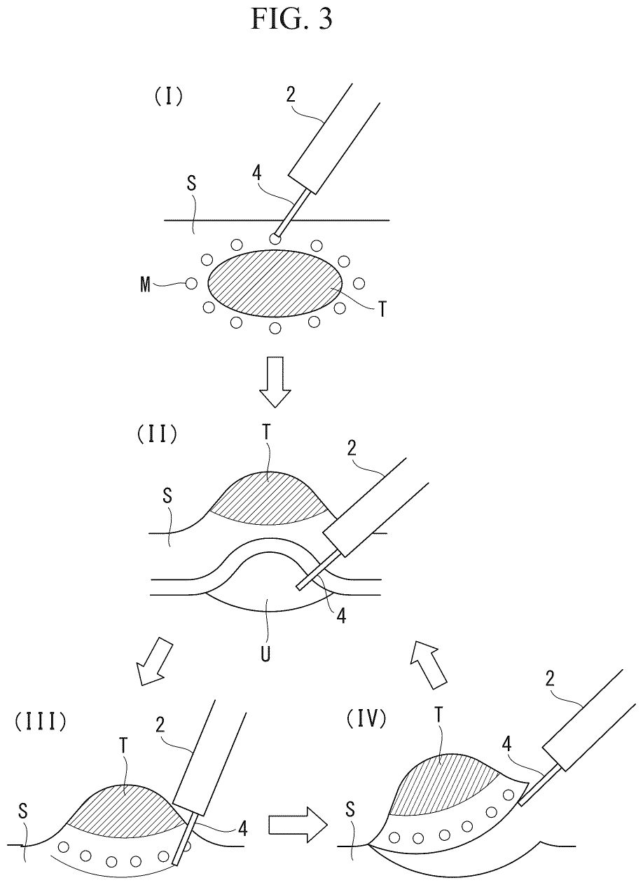

FIG. 3 illustrates typical steps in an endoscopic submucosal dissection (ESD) of the stomach (subject) S, which is an example of an endoscopic procedure to which the image processing method is applied.

In an ESD, markings M, indicating a range to be resected, are provided around the lesion T (step I). FIG. 3 illustrates multiple dotted markings M arranged in a circle. Next, a drug U is locally injected into the submucosal layer in order to raise the lesion T (step II), the mucosa is incised along the outside of the markings M by the treatment tool 4, such as a knife (step III), and the lesion T is dissected (step IV).

The local injection, the incision, and the dissection are repeated multiple times until the entire lesion T is dissected.

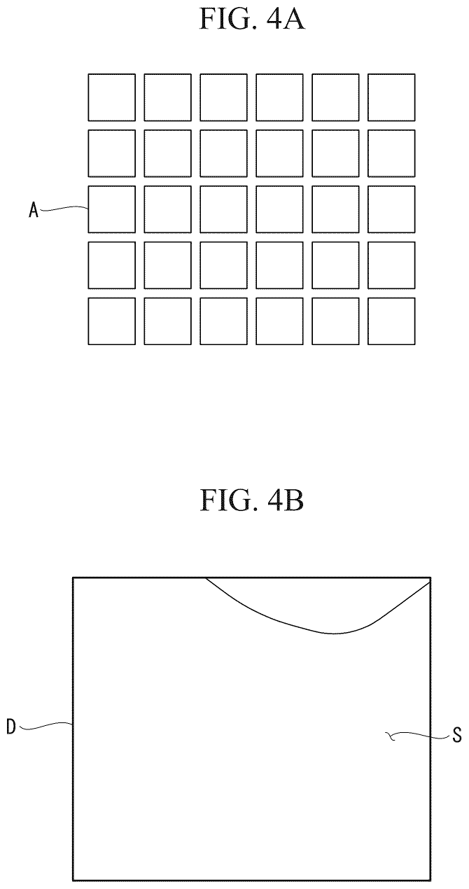

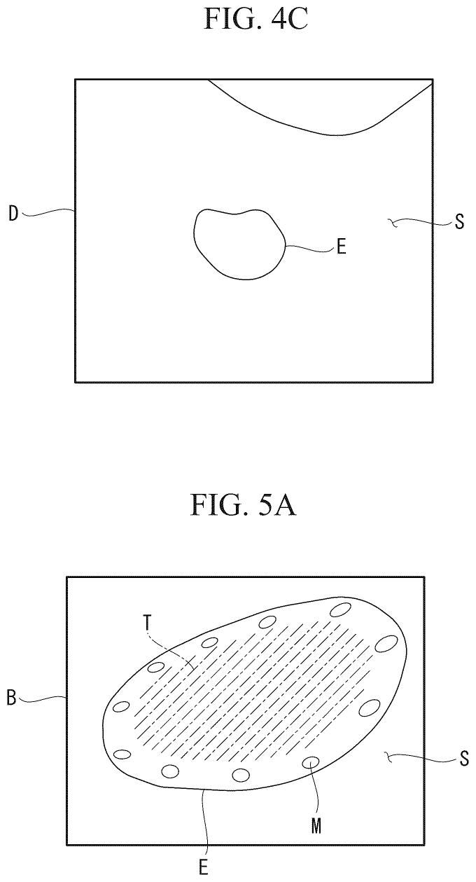

As illustrated in FIG. 4A, the processor 11 acquires a sequence of two-dimensional images of the stomach S as the first images A (step S1). Typically, an endoscopic examination of the stomach S is performed before the ESD procedure. The first images A are images acquired by the endoscope 2 during the endoscopic examination. For example, images at the time of the endoscopic examination are stored in the storage unit 13, and the processor 11 acquires the first images A by reading the first images A from the storage unit 13. The endoscope 2 used in the endoscopic examination may be the same as or different from the endoscope 2 used in the endoscopic procedure.

Next, as illustrated in FIG. 4B, the processor 11 generates a 3D image D of the stomach S from the sequence of first images A using a known three-dimensional reconstruction technology, such as simultaneous localization and mapping (SLAM) or structure from motion (SfM) (step S2). In the three-dimensional reconstruction process for generating the 3D image D, the position of the camera 2a corresponding to each of the first images A in the 3D image D is calculated. The position of the camera 2a may be stored in the storage unit 13 for use in step S6 described below.

After the start of the procedure, the processor 11 generates the boundary information E on the second image B, calculates the position of the boundary information E on the second image B, and associates the calculated position with the second image B (step S3). The second image B is a two-dimensional image of the stomach S acquired by the endoscope 2 during the procedure. The boundary information E is information indicating the position of the boundary of the lesion T to be resected.

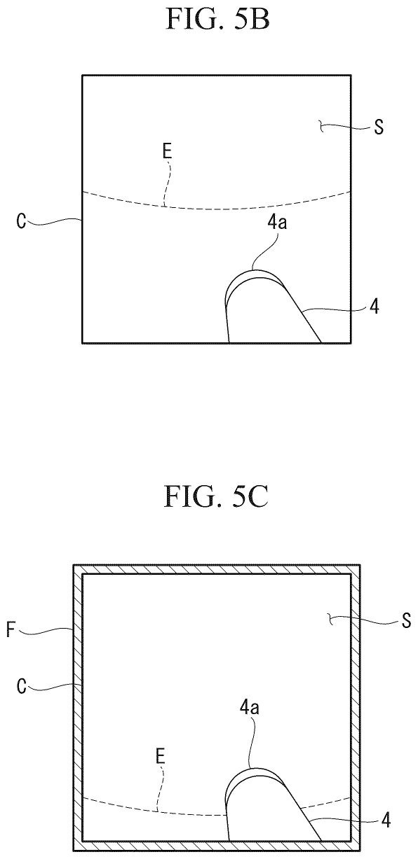

For example, as illustrated in FIG. 5A, the processor 11 generates an incision line, serving as the boundary information E, along the boundary of the lesion T based on the input by the user. The user marks, on the second image B having a wide field of view including the entirety of the lesion T and the markings M, the incision line E surrounding the row of markings M using the user interface 14. The processor 11 captures the second image B marked with the incision line E, and calculates the position of the incision line E in the second image B.

The processor 11 may generate the boundary information E by another method. For example, the processor 11 may detect the markings M from the second image B having a wide field of view and generate a row of markings M as the boundary information.

Next, as illustrated in FIG. 4C, the processor 11 associates the position of the boundary information E, associated with the second image B, with the 3D image D (step S4). For example, the processor 11 performs matching between the second image B and the 3D image D to calculate the position of the incision line E in the 3D image D.

After step S4, the processor 11 receives, in real time, an image input from the endoscope 2 to the image processing apparatus 1 as the third image C (step S5). Hence, the third image C is a two-dimensional image acquired by the endoscope 2 after the second image B. The processor 11 executes steps S6 to S8 using the current third image C.

As illustrated in FIG. 5B, when a mucosal incision is started, the treatment tool 4 is disposed within the field of view of the endoscope 2. The processor 11 calculates the position of a distal end 4a of the treatment tool 4 in the 3D image D using the third image C, based on the characteristic that the relative position between the treatment tool 4 and the camera 2a is constant (step S6).

FIG. 2B illustrates a specific example of step S6.

During the treatment, the treatment tool 4 moves in unison with the endoscope 2. Hence, the background region (i.e., the region other than the treatment tool 4) in the third image C moves across multiple third images C, whereas the treatment tool 4 does not move across multiple third images C. The processor 11 detects a stationary region that is stationary across at least two sequential third images C as the treatment tool 4 (step S61).

Next, the processor 11 calculates the relative position between the camera 2a and the distal end 4a based on the position of the treatment tool 4 in the third image C and the optical design data of the endoscope 2 (step S62).

Next, the processor 11 calculates the current position of the camera 2a in the 3D image D based on the third image C (step S63). For example, the processor 11 performs matching between the third image C and the 3D image D to calculate the position of the third image C in the 3D image D, and calculates the position of the camera 2a in the 3D image D based on the position of the third image C. The processor 11 may calculate the position of the camera 2a in the 3D image D using the position of the camera 2a calculated in step S2.

Next, the processor 11 calculates the position of the distal end 4a in the 3D image D based on the relative position calculated in steps S61 and S62 and the position of the camera 2a (step S64).

Next, the processor 11 determines whether or not the position of the treatment tool 4 with respect to the incision line E is appropriate, for example, whether the distal end 4a is on the outside or inside of the incision line E, based on the position of the boundary information E associated with the 3D image D in step S4 and the position of the distal end 4a calculated in step S6 (step S7).

Next, the processor 11 outputs support information based on the determination result in step S7 (step S8). The support information is information for supporting the operation of the treatment tool 4 by the surgeon so that the position or movement direction of the treatment tool 4 is appropriate with respect to the incision line E. The surgeon can recognize whether or not the operation of the treatment tool 4 with respect to the incision line E is appropriate based on the support information.

As illustrated in FIG. 5C, an example of the support information is an alert indicating that the distal end 4a is inside the incision line E. When the distal end 4a is inside the incision line E, the processor 11 displays an alert F on the display device 3 or outputs an alert sound from a speaker. In FIG. 5C, a frame indicating the alert F is displayed on the edge of the third image C. In contrast, when the distal end 4a is outside the incision line E, the processor 11 does not output an alert (see FIG. 5B). The surgeon recognizes that the distal end 4a is inside the incision line E based on the output of the alert, and corrects the position of the distal end 4a in the direction away from the lesion T.

In an ESD, it is important to incise the outside of the boundary of the lesion T in order to reliably resect the entire lesion T. However, typically, the boundary of the lesion T in the stomach is often unclear. In addition, because the portion to be incised is magnified and observed at the time of incision, the field of view of the third image C is narrow. Thus, it may be difficult to recognize the position of the distal end 4a with respect to the markings M or the lesion T based only on the third image C.

According to this embodiment, the position of the treatment tool 4 with respect to the incision line E is determined, and, when the treatment tool 4 is disposed at an inappropriate position with respect to the incision line E, for example, when the distal end 4a is positioned within the incision line E, the surgeon is notified of that fact by the support information F, such as an alert. In this way, it is possible to support the operation of the treatment tool 4 by the surgeon so as to reliably incise the outside of the incision line E.

According to this embodiment, the 3D image D is generated from the two-dimensional first images A acquired by a typical monocular endoscope 2 before the endoscopic procedure. Hence, it is not necessary to perform an inspection with a three-dimensional image diagnostic apparatus, such as a CT apparatus or an MRI apparatus, before the endoscopic procedure. In addition, the use of a special endoscope in the endoscopic examination is not required.

According to this embodiment, the current positions of the camera 2a and the distal end 4a in the 3D image D during the endoscopic procedure are calculated by using the two-dimensional second image B. Thus, a sensor for detecting the positions of the camera 2a and the treatment tool 4 is not required. Accordingly, the image processing method described above can be realized using the endoscope 2 and the treatment tool 4 that are commonly used in endoscopic procedures.

Conventionally, there is a known technology that uses sensors mounted on an endoscope and a treatment tool to detect the current positions of the endoscope and the treatment tool in the body (for example, see Japanese Unexamined Patent Application, Publication Nos. 2013-202313 and 2014-230612). This technology cannot be applied to typical endoscopes and typical treatment tools without sensors.

In this embodiment, the processor 11 may correct a positional deviation of the treatment tool 4 in the third image C due to distortion of the third image C and calculate the position of the distal end 4a in the 3D image D using the corrected position of the treatment tool 4.

FIG. 2C illustrates an example of step S6 including correction of the position of the treatment tool 4.

The processor 11 calculates, based on a distortion correction coefficient and the optical design data of the endoscope 2, the offset amounts at the respective positions in the third image C due to distortion, and generates a conversion table based on the offset amounts (step S65). The conversion table is a table for converting the respective positions in the third image C into corrected positions. Step S65 may be executed at any timing before step S63, and the conversion table may be stored in the storage unit 13 in advance.

The processor 11 calculates, using the conversion table, a corrected position of the treatment tool 4 based on the position of the treatment tool 4 detected in step S61 (step S66), and calculates a relative position between the camera 2a and the distal end 4a based on the corrected position of the treatment tool 4 (step S62).

With this configuration, the calculation of the position of the distal end 4a in the 3D image D and the determination of the positional relationship between the treatment tool 4 and the incision line E can be performed with higher accuracy.

### Second Embodiment

Next, an image processing apparatus, an image processing method, and an image processing program according to a second embodiment of the present invention will be described with reference to the drawings.

This embodiment differs from the first embodiment in that the image processing apparatus 1 provides a surgeon with navigation information for navigating the direction of incision by the treatment tool 4 during a procedure. In this embodiment, the configurations different from those in the first embodiment will be described, and the configurations common to those in the first embodiment will be denoted by the same reference signs, and a description thereof will be omitted.

The image processing apparatus 1 according to this embodiment is applied to the endoscope system 100 and includes the processor 11, the memory 12, the storage unit 13, and the user interface 14, similarly to the first embodiment.

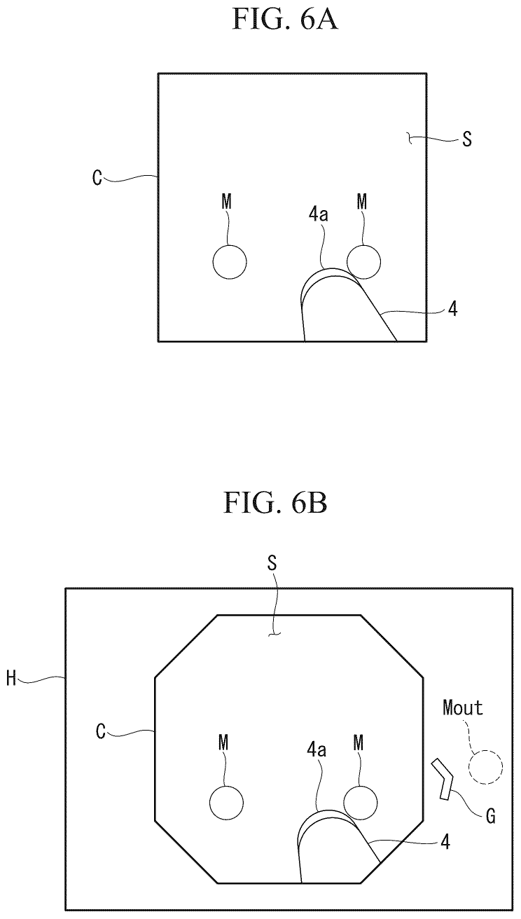

As described above, in a gastric ESD, multiple markings M arranged at intervals are provided, and then the mucosa is incised outside the row of markings M. At the time of incision, the surgeon brings the distal end of the endoscope 2 close to the mucosa in order to magnify and observe the portion to be incised. Hence, as illustrated in FIG. 6A, the field of view of the third image C at the time of incision is narrow, and there may be only one or two markings M or no markings M in the third image C. In such a case, the surgeon may not know the direction in which the incision is to be made.

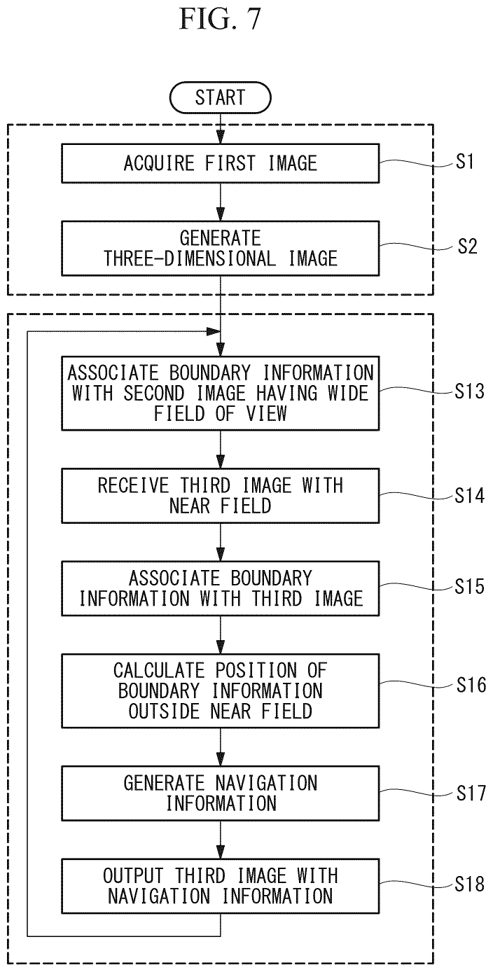

FIG. 7 illustrates an image processing method according to this embodiment, executed by the processor 11.

The image processing method includes step S1, step S2, step S13 of associating the position of the boundary information E of the lesion T with the second image B of the subject S, step S14 of receiving the third image C of the subject S, step S15 of associating the position of the boundary information E with the third image C, step S16 of calculating the position of the boundary information E outside the field of view of the third image C, step S17 of generating navigation information G, and step S18 of outputting the third image C together with the navigation information G.

Steps S1 and S2, which have been described in the first embodiment, are executed before the procedure using the first images A acquired at the time of the endoscopic examination. Steps S13 to S18 are performed during the ESD procedure.

The second image B is an image having a wide field of view including the entirety of the lesion T and the markings M. The processor 11 detects the multiple markings M arranged in a circle in the second image B as the boundary information E using, for example, a known image recognition technology, and calculates the positions of the markings M in the second image B (step S13). The processor 11 may determine the second image B from which the markings M are detected based on, for example, an input by a user, such as a surgeon.

After step S13, the processor 11 receives, in real time, an image input from the endoscope 2 to the image processing apparatus 1 as the third image C (step S14), and executes steps S15 to S18 using the current third image C. At the time of mucosal incision, the surgeon brings the distal end of the endoscope 2 close to the markings M to magnify and observe the portion to be incised. The third image C is an image with a near field, which is narrower than the field of view of the second image B.

The processor 11 associates the positions of the markings M in the second image B with the third image C via the 3D image D (step S15) to calculate the positions of markings Mout located outside the field of view (near field) of the third image C (step S16).

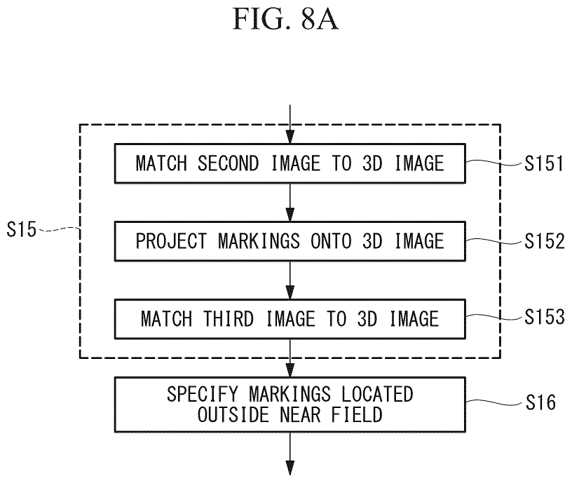

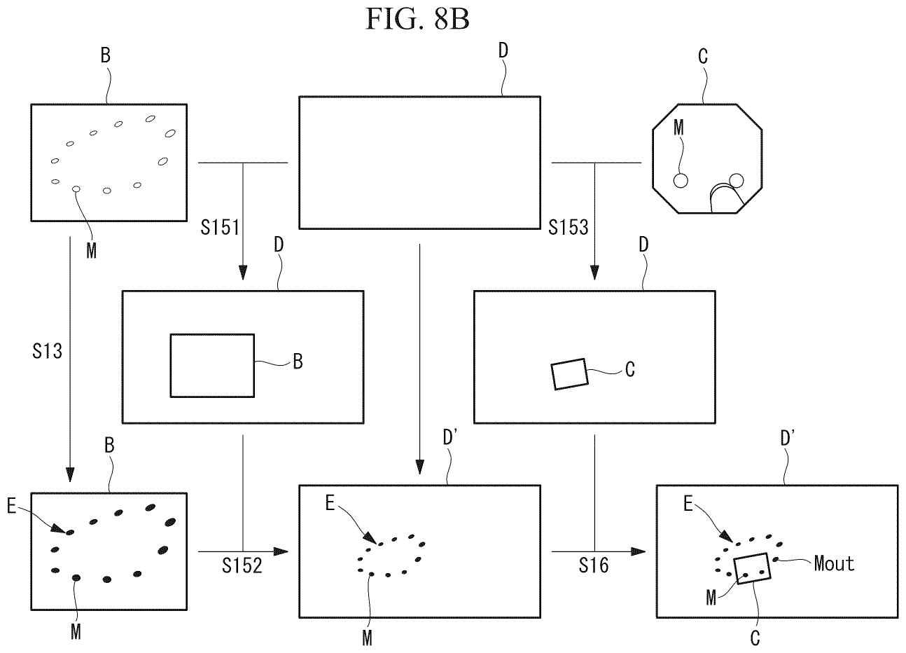

FIGS. 8A and 8B illustrate a first example of steps S15 and S16.

The processor 11 associates the position of the boundary information E in the second image B with the 3D image D (S152, S152). More specifically, the processor 11 performs matching between the second image B and the 3D image D to calculate the position of the second image B in the 3D image D (step S151). Next, the processor 11 projects the markings M detected in step S13 onto the 3D image D based on the position calculated in step S151, and calculates the positions of the markings M in the 3D image D (step S152).

Next, the processor 11 associates the position of the third image C with the 3D image D. More specifically, the processor 11 performs matching between the third image C and the 3D image D to calculate the position of the third image C in the 3D image D (step S153). The position of the boundary information E is associated with the third image C on the 3D image D in steps S151, S152, and S153.

Next, the processor 11 specifies the markings Mout located outside the third image C with a near field and adjacent to the near field based on the positions of the markings M and the position of the third image C calculated in steps S152 and S153 (step S16).

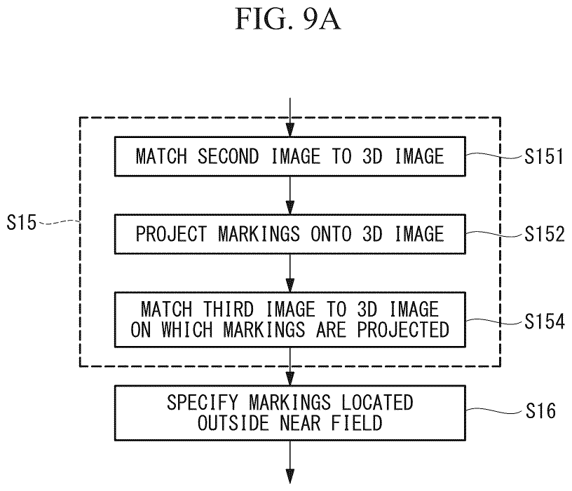

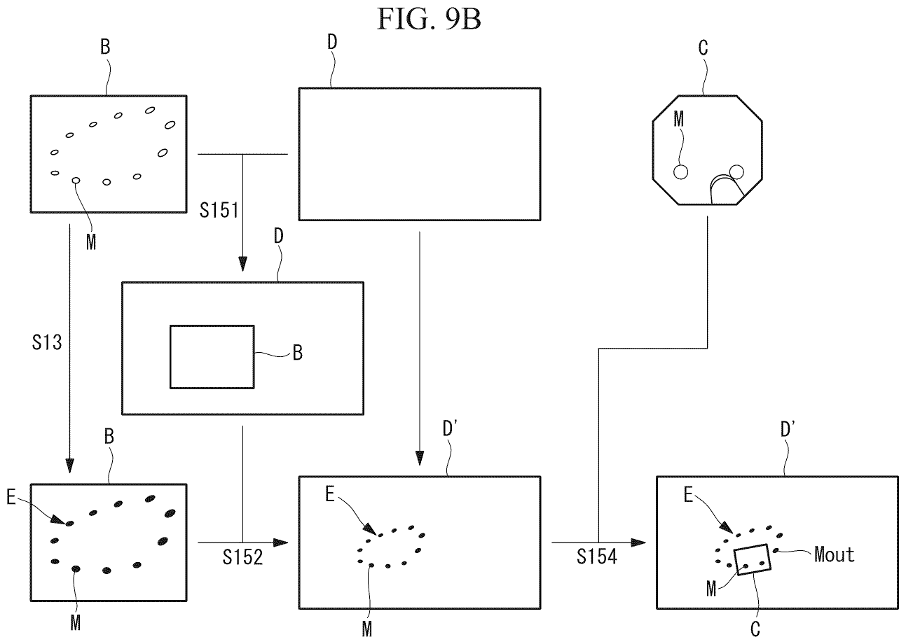

FIGS. 9A and 9B illustrate a second example of steps S15 and S16.

The processor 11 associates the position of the boundary information E in the second image B with the 3D image D (S152, S152), as in the first example.

Next, the processor 11 associates the position of the third image C with a 3D image D′ on which the markings M are projected and with which the position of the boundary information E is associated (step S154). More specifically, the processor 11 performs matching between the third image C and the 3D image D′ to calculate the position of the third image C in the 3D image D′. Through steps S151, S152, and S154, the position of the boundary information E is associated with the third image C on the 3D image D′.

Next, the processor 11 specifies the markings Mout based on the positions of the markings M and the position of the third image C calculated in steps S152 and S154 (step S16).

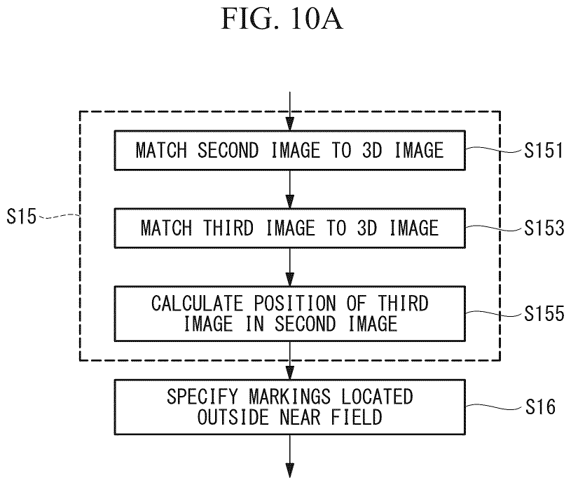

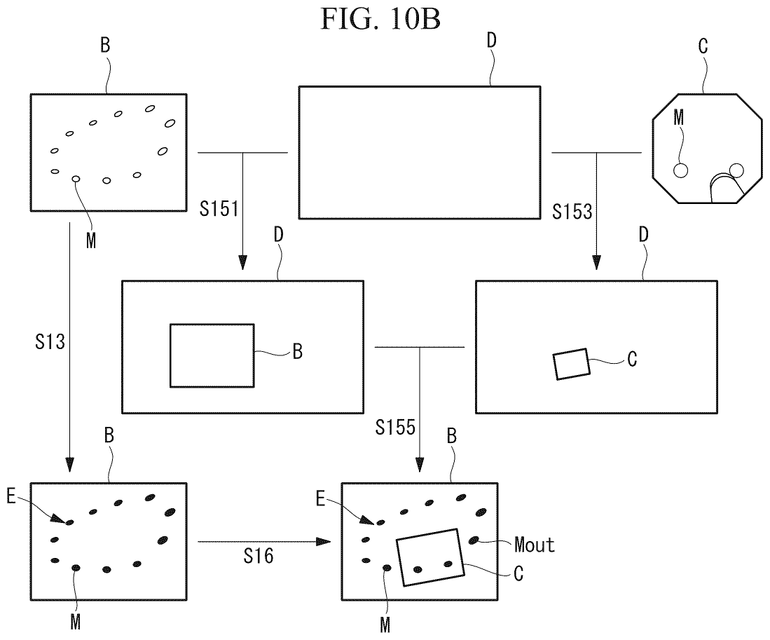

FIGS. 10A and 10B illustrate a third example of steps S15 and S16.

The processor 11 associates the position of the third image C with the second image B via the 3D image D. For example, the processor 11 performs steps S151 and S153 as in the first example. The position of the boundary information E is associated with the third image C through steps S151 and S153.

Next, the processor 11 calculates the position of the third image C in the second image B based on the positions of the images B and C calculated in steps S151 and S153 (step S155).

Next, the processor 11 specifies the markings Mout based on the positions of the markings M and the position of the third image C calculated in steps S13 and S155 (step S16).

The matching in steps S151, S153, and S154 may be performed based on landmarks, which are feature points in the images B, C, and D. For example, the processor 11 may detect the position, type, and feature value of one or more landmarks from each of the images B, C, and D, create a proximity graph from the landmarks for each of the images B, C, and D, and perform matching between the images using the proximity graph.

Subsequent to step S16, the processor 11 generates the navigation information G (step S17) and outputs the third image C together with the navigation information G to the display device 3 to be displayed (step S18). The navigation information G is an indication of the position or direction of the markings Mout calculated in step S16, and is, for example, an indication, such as an arrow, pointing to the markings Mout or an indication disposed at the position of the markings Mout. As illustrated in FIG. 6B, the processor 11; may generate a composite image H in which the navigation information G is overlaid on the outside of the third image C, and display the composite image H on the display device 3. In FIG. 6B, the indication G indicating the direction is disposed between a marking M in the third image C and a marking Mout.

As illustrated in FIG. 6A, when there are only two markings M in the third image C, it is difficult for the surgeon to determine, from only the third image C, in which direction the incision should be made after an incision is made from the marking M on the left side to the marking M on the right side.

According to this embodiment, the surgeon can grasp the boundary information E outside the field of view of the third image C based on the navigation information G outside the third image C, and recognize the direction in which an incision is to be made.

Furthermore, according to this embodiment, similarly to the first embodiment, there is no need to perform an examination with a three-dimensional image diagnostic apparatus, such as a CT apparatus or an MRI apparatus, before an endoscopic procedure, and there is no need to use a special endoscope in an endoscopic examination. Furthermore, according to this embodiment, the image processing method described above can be realized using the endoscope 2 and the treatment tool 4 that are commonly used in endoscopic procedures.

### Third Embodiment

Next, an image processing apparatus, an image processing method, and an image processing program according to a third embodiment of the present invention will be described with reference to the drawings.

This embodiment differs from the first embodiment in that the image processing apparatus 1 provides a surgeon with progress information showing the progress of a treatment by the treatment tool 4 during a procedure. In this embodiment, the configurations different from those in the first embodiment will be described, and the configurations common to those in the first embodiment will be denoted by the same reference signs, and the description thereof will be omitted.

The image processing apparatus 1 according to this embodiment is applied to the endoscope system 100 and includes the processor 11, the memory 12, the storage unit 13, and the user interface 14, similarly to the first embodiment.

As described above, in a gastric ESD, the resection of the lesion T is advanced stepwise by repeating local drug injection, mucosal incision, and dissection of the lesion T. It is sometimes difficult for an unskilled surgeon to determine the timing of proceeding from incision to dissection. In addition, the larger the lesion T, the more difficult for the surgeon to grasp the progress of the treatment.

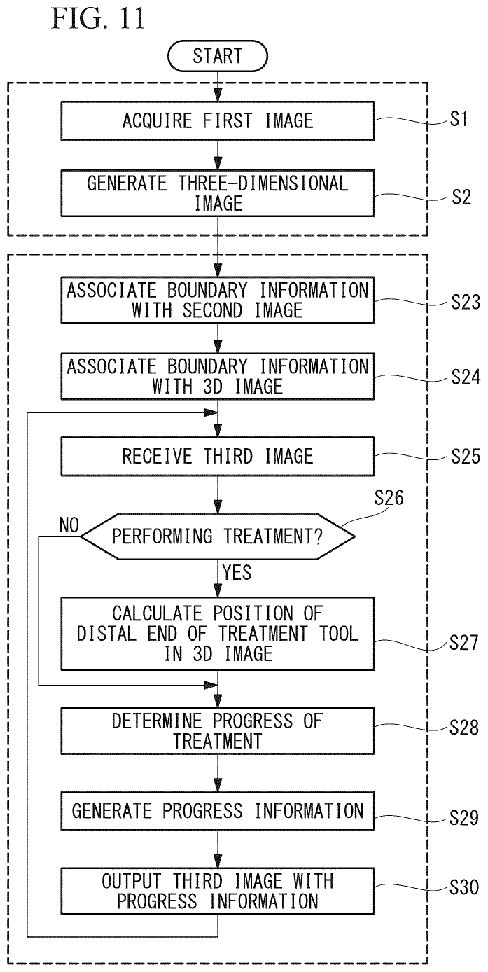

FIG. 11 illustrates an image processing method according to this embodiment, executed by the processor 11.

The image processing method includes step S1, step S2, step S23 of associating the position of the boundary information E of the lesion T with the second image B of the subject S, step S24 of associating the position of the boundary information E with the 3D image D, step S25 of receiving the third image C of the subject S, step S26 of determining whether or not the treatment tool 4 in the third image C is performing a treatment, step S27 of calculating the position of the treatment tool 4 in the 3D image D based on the third image C, step S28 of determining the progress of the treatment, step S29 of generating progress information I, and step S30 of outputting the third image C together with the progress information I.

Steps S1 and S2, which have been described in the first embodiment, are executed before the procedure using the first images A acquired at the time of the endoscopic examination. Steps S23 to S30 are performed during the ESD procedure.

Steps S23 and S24 are identical to steps S3 and S4 in the first embodiment, respectively. The boundary information E is an incision line to be incised by the treatment tool 4.

After step S24, the processor 11 receives, in real time, the third image C input from the endoscope 2 to the image processing apparatus 1 (step S25), and executes steps S26 to S30 using the current third image C.

The processor 11 determines whether or not the treatment tool 4 is performing a treatment based on at least the position of the treatment tool 4 in the third image C (step S26). For example, in the case where the treatment tool 4 is an energy device, such as an electric scalpel, the mucosa is incised only when the treatment tool 4 is powered and the treatment tool 4 is in contact with the mucosa. The processor 11 determines that the treatment is being performed when the treatment tool 4 is in contact with the mucosa and the treatment tool 4 is powered. Hence, the processor 11 may acquire, as necessary, information indicating whether or not the treatment tool 4 is in contact with the mucosa and information indicating whether or not the treatment tool 4 is powered in step S26.

When it is determined that the treatment tool 4 is not performing a treatment (NO in step S26), the processor 11 proceeds to step S28.

When it is determined that the treatment tool 4 is performing a treatment (YES in step S26), the processor 11 calculates the position of the distal end 4a in the third image C at that time based on the third image C (step S27). The calculation of the position of the distal end 4a in the 3D image D is performed in the same manner as in step S6 in the first embodiment. The processor 11 may calculate the position of the distal end 4a using another method. For example, the processor 11 may detect the treatment tool 4 in the third image C using a known image recognition technology and calculate the position of the distal end 4a in the 3D image D by matching between the third image C and the 3D image D.

Next, the processor 11 determines the progress of the treatment based on the position of the incision line E associated with the 3D image D in step S23 and the position of the distal end 4a calculated in step S27 (step S28). For example, the processor 11 accumulates the position of the distal end 4a calculated in step S27 to obtain information on the position of the incision that has already been made. The processor 11 determines the portion of the incision line E that has already been incised, based on the comparison between the position of the accumulated distal end 4a and the position of the incision line E.

Next, the processor 11 generates the progress information I indicating the progress of the incision (step S29).

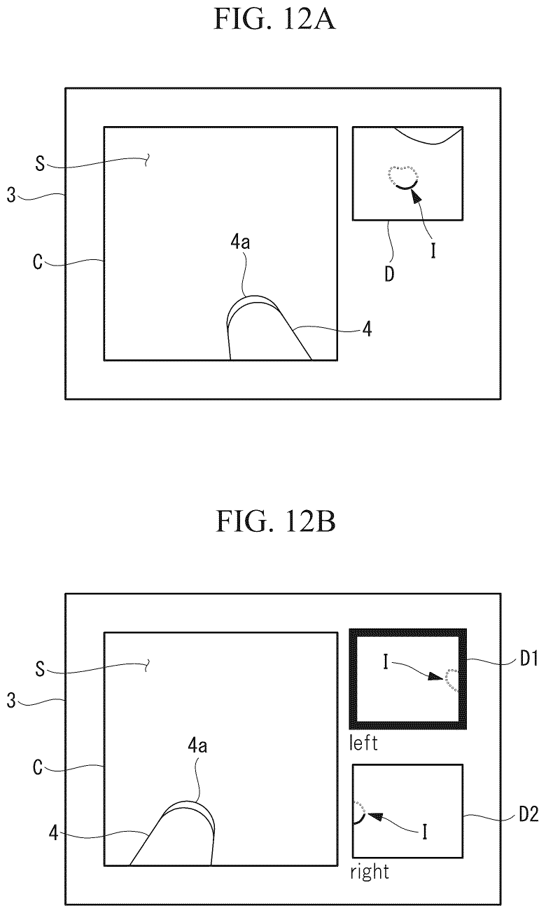

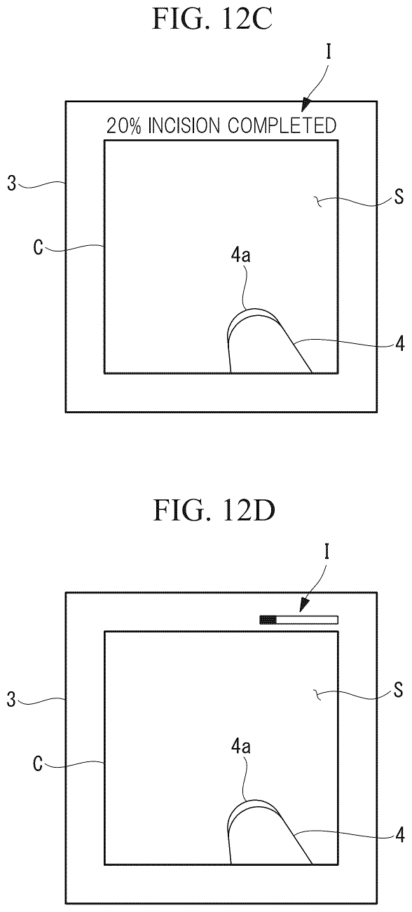

As illustrated in FIG. 12A, an example of the progress information I is a figure representing the incision line E. The progress information may be a 3D image D in which the figure I is superimposed at the position of the incision line E. In the figure I, the already incised portion and the portion that has not yet been incised are displayed in mutually different manners (e.g., in different colors). In FIG. 12A, the already incised portion is indicated by a solid line and the portion that has not yet been incised is indicated by a dotted line to show the difference.

As illustrated in FIG. 12B, the 3D image D including the figure I may be divided into multiple regions D1 and D2. In this case, the processor 11 selects one region including the position of the distal end 4a from the multiple regions D1 and D2, and displays the selected region so as to be distinguishable from the other region. In FIG. 12B, a horizontally long 3D image D is divided into a left area D1 and a right area D2, and a frame is added to the selected left area D1. According to this configuration, in the case of a large lesion T or the like, the surgeon can easily compare the third image C with the current incision position in the figure I.

As illustrated in FIG. 12C, another example of the progress information I is an indication including a numerical value representing the proportion of the length of the portion that has been incised in the overall length of the incision line E.

As illustrated in FIG. 12D, another example of the progress information I is a progressing bar.

Next, the processor 11 outputs the third image C together with the progress information I to the display device 3 to be displayed (step S30). For example, as illustrated in FIGS. 12A to 12D, the progress information I is displayed on the display device 3 side by side with the third image C.

As described above, during an ESD procedure, it is difficult to grasp the progress of the treatment based only on the third image C with a near field.

According to this embodiment, the surgeon can know the progress of the incision based on the progress information I. Accordingly, the surgeon can smoothly proceed with the treatment by determining the timing of proceeding to the next step, such as dissection, and can perform an appropriate treatment.

Furthermore, according to this embodiment, similarly to the first embodiment, there is no need to perform an examination with a three-dimensional image diagnostic apparatus, such as a CT apparatus or an MRI apparatus, before an endoscopic procedure, and there is no need to use a special endoscope in an endoscopic examination. Furthermore, according to this embodiment, the image processing method described above can be realized using the endoscope 2 and the treatment tool 4 that are commonly used in endoscopic procedures.

Although the embodiments of the present invention and the modifications thereof have been described above, the present invention is not limited thereto, and can be appropriately modified without departing from the gist of the present invention.

For example, the image processing apparatus and the image processing method according to the present invention can be applied to other endoscopic procedures in which a tissue is incised along a boundary of a lesion, in addition to ESD procedures.

The first image A is not limited to an image acquired at the time of an endoscopic examination, and may be a two-dimensional image acquired by the endoscope 2 at any timing before the second image B. For example, in an endoscopic procedure, the processor 11 may acquire a sequence of first images A of a subject, generate a 3D image D, and then execute processing using a second image B.

### REFERENCE SIGNS LIST

1 Image processing apparatus
2 Endoscope
2a Camera
3 Display device
4 Treatment tool
4a Distal end
11 Processor
13 Storage unit
13a Image processing program
A First image
B Second image
C Third image
D Three-dimensional image
E Incision line (boundary information)
F Alert (support information)
G Navigation information
H Composite image
I Progress information
S Subject
T Lesion
M Marking

1. An image processing apparatus that processes an image acquired by an endoscope, the apparatus comprising a processor configured to:
generate a three-dimensional image of a subject from a first image;
associate a position of boundary information of a lesion included in a second image with the second image; and
associate the position of the boundary information associated with the second image with the three-dimensional image,
wherein the first image and the second image are two-dimensional images of the subject acquired by the endoscope, and the first image is acquired before an acquisition of the second image, and
wherein the processor is further configured to:
calculate a relative position between a treatment tool in a third image and a camera of the endoscope based on the third image, the third image being a two-dimensional image acquired by the endoscope after the acquisition of the second image;
calculate a position of the camera in the three-dimensional image based on the third image;
calculate a position of the treatment tool in the three-dimensional image based on the relative position and the position of the camera in the three-dimensional image;
determine a positional relationship between the treatment tool and the boundary information based on the position of the boundary information associated with the three-dimensional image and the position of the treatment tool in the three-dimensional image; and
output support information based on a determination result.

2. The image processing apparatus according to claim 1, wherein the processor is configured to calculate the position of the camera in the three-dimensional image using the position of the camera calculated in generating the three-dimensional image.

3. The image processing apparatus according to claim 1, wherein the processor is configured to detect a region that remains stationary across a sequence of third images as the treatment tool in calculating the relative position.

4. The image processing apparatus according to claim 1, wherein the processor is configured to:
correct a positional deviation of the treatment tool in the third image due to distortion; and
calculate the relative position based on the corrected position of the treatment tool.

5. An image processing apparatus that processes an image acquired by an endoscope, the apparatus comprising a processor configured to:
generate a three-dimensional image of a subject from a first image;
associate a position of boundary information of a lesion included in a second image with the second image; and
associate the position of the boundary information associated with the second image with the three-dimensional image,
wherein the first image and the second image are two-dimensional images of the subject acquired by the endoscope, and the first image is acquired before an acquisition of the second image, and
wherein the processor is further configured to:
associate the position of the boundary information with the second image by detecting the boundary information in the second image, the second image having a wide field of view including an entirety of the lesion;
associate the position of the boundary information in the second image with a third image via the three-dimensional image, the third image being a two-dimensional image acquired by the endoscope after the acquisition of the second image and being a two-dimensional image having a narrower field of view than the second image;
calculate the position of the boundary information outside the field of view of the third image; and
output the third image together with navigation information relating to the boundary information outside the field of view.

6. The image processing apparatus according to claim 5, wherein the processor is configured to:
associate a position of the third image with the three-dimensional image; and
calculate the position of the boundary information outside the field of view based on the position of the boundary information associated with the three-dimensional image and the position of the third image.

7. The image processing apparatus according to claim 6, wherein the processor is configured to:
detect a feature point from each of the three-dimensional image, the second image, and the third image;
associate a position of the second image with the three-dimensional image based on the feature point in the second image and the feature point in the three-dimensional image; and
associate the position of the third image with the three-dimensional image based on the feature point in the third image and the feature point in the three-dimensional image.

8. The image processing apparatus according to claim 5, wherein the navigation information is an indication of a position or a direction of the boundary information located outside and adjacent to the field of view of the third image.

9. An image processing apparatus that processes an image acquired by an endoscope, the apparatus comprising a processor configured to:
generate a three-dimensional image of a subject from a first image;
associate a position of boundary information of a lesion included in a second image with the second image; and
associate the position of the boundary information associated with the second image with the three-dimensional image,
wherein the first image and the second image are two-dimensional images of the subject acquired by the endoscope, and the first image is acquired before an acquisition of the second image,
wherein the boundary information is a line to be treated by a treatment tool, and
wherein the processor is further configured to:
determine whether or not the treatment tool in a third image is performing a treatment, the third image being a two-dimensional image acquired by the endoscope after the acquisition of the second image;
calculate, when it is determined that the treatment tool is performing a treatment, a position of the treatment tool in the three-dimensional image at that time based on the third image;
determine progress of the treatment based on a position of the line associated with the three-dimensional image and the position of the treatment tool in the three-dimensional image; and
output the third image together with progress information indicating the progress of the treatment.

10. The image processing apparatus according to claim 9, wherein the progress information is a figure representing the line, and in the figure, a portion that has been treated and a portion that has not yet been treated are displayed in different manners.

11. The image processing apparatus according to claim 9, wherein the progress information includes a numerical value representing a proportion of a length of the portion that has been treated in an overall length of the line.

12. An image processing method for processing an image acquired by an endoscope, the method comprising:
generating a three-dimensional image of a subject from a first image;
associating a position of boundary information of a lesion included in a second image with the second image; and
associating the position of the boundary information associated with the second image with the three-dimensional image,
wherein the first image and the second image are two-dimensional images of the subject acquired by the endoscope, and the first image is acquired before an acquisition of the second image, and
wherein the method further comprises:
calculating a relative position between a treatment tool in a third image and a camera of the endoscope based on the third image, the third image being a two-dimensional image acquired by the endoscope after the acquisition of the second image;
calculating a position of the camera in the three-dimensional image based on the third image;
calculating a position of the treatment tool in the three-dimensional image based on the relative position and the position of the camera in the three-dimensional image;
determining a positional relationship between the treatment tool and the boundary information based on the position of the boundary information associated with the three-dimensional image and the position of the treatment tool in the three-dimensional image; and
outputting support information based on a determination result.

13. A non-transitory computer-readable recording medium having an image processing program stored therein, the program causing a processor to execute an image processing method for processing an image acquired by an endoscope, the image processing method comprising:
generating a three-dimensional image of a subject from a first image;
associating a position of boundary information of a lesion included in a second image with the second image; and
associating the position of the boundary information associated with the second image with the three-dimensional image,
wherein the first image and the second image are two-dimensional images of the subject acquired by the endoscope, and the first image is acquired before an acquisition of the second image, and
wherein the method further comprises:
calculating a relative position between a treatment tool in a third image and a camera of the endoscope based on the third image, the third image being a two-dimensional image acquired by the endoscope after the acquisition of the second image;
calculating a position of the camera in the three-dimensional image based on the third image;
calculating a position of the treatment tool in the three-dimensional image based on the relative position and the position of the camera in the three-dimensional image;
determining a positional relationship between the treatment tool and the boundary information based on the position of the boundary information associated with the three-dimensional image and the position of the treatment tool in the three-dimensional image; and
outputting support information based on a determination result.
